SEQUENCE LISTING (15 sequences)

SEQ ID NO: 1 - DNA, 20 bp, Artificial Sequence
  Exon 2 Sense Primer Sequence
ttaggggtccagtcatatgc

SEQ ID NO: 2 - DNA, 20 bp, Artificial Sequence
  Exon 2 Antisense Primer Sequence
cctgaagtcactatcaggca

SEQ ID NO: 3 - DNA, 20 bp, Artificial Sequence
  Exon 3 Sense Primer Sequence
cggcacagccacacccgact

SEQ ID NO: 4 - DNA, 20 bp, Artificial Sequence
  Exon 3 Antisense Primer Sequence
tgtagaggcatagggcatag

SEQ ID NO: 5 - DNA, 22 bp, Artificial Sequence
  Exon 4 Sense Primer Sequence
ctcctgcagtaatggctttgtc

SEQ ID NO: 6 - DNA, 22 bp, Artificial Sequence
  Exon 4 Antisense Primer Sequence
tcagggtcagggatctcaaggc

SEQ ID NO: 7 - DNA, 19 bp, Artificial Sequence
  Exon 5 Sense Primer Sequence
ctgccccgagcctgactcc

SEQ ID NO: 8 - DNA, 19 bp, Artificial Sequence
  Exon 5 Antisense Primer Sequence
tggtcatgggggtggggcc

SEQ ID NO: 9 - DNA, 20 bp, Artificial Sequence
  Nbk Sense Primer Sequence
gtctgaagtaagacccctct

SEQ ID NO: 10 - DNA, 20 bp, Artificial Sequence
  Nbk Antisense Primer Sequence
acttgagcagcaggtgcagg

SEQ ID NO: 11 - DNA, 30 bp, Artificial Sequence
  Beta-actin Sense Primer Sequence
acccccaaggccaaccgcgagaagatgacc

SEQ ID NO: 12 - DNA, 30 bp, Artificial Sequence
  Beta-Actin Antisense Primer Sequence
ggtgatgacctggccgtcaggcagctcgta

SEQ ID NO: 13 - DNA, 22 bp, Artificial Sequence
  Real time PCR Sense Primer Sequence
ttaagtgtggtgaaaccgtcca

SEQ ID NO: 14 - DNA, 19 bp, Artificial Sequence
  Real-time PCR Antisense Primer Sequence
cacagcctgggtctggctt

SEQ ID NO: 15 - DNA, 27 bp, Artificial Sequence
  Fluorsescent Probe
catccctgatgtcctcagtctggtcgt

(19) United States
(12) Patent Application Publication
(10) Pub. No.: US 2005/0053931 A1
(43) Pub. Date: Mar. 10, 2005

(54) Method for the diagnosis and therapy of renal cell carcinoma

(76) Inventors: Peter Daniel, Berlin (DE); Bernhard Gillissen, Berlin (DE)

Correspondence Address: BIRCH STEWART KOLASCH & BIRCH, PO BOX 747, FALLS CHURCH, VA, 22040-0747, US

(21) Appl. No.: 10/479,200

(22) PCT Filed: May 30, 2002
(86) PCT No.: PCT/DE02/01999

(30) Foreign Application Priority Data
May 31, 2001 (DE) 101 26 473.9

(51) Int. Cl. C12Q 1/68; G01N 33/53; G01N 33/567

(52) U.S. Cl. 435/6; 435/7.2

(57) ABSTRACT

The invention relates to a novel approach for the diagnosis and therapy of renal cell carcinoma, and other renal tumors, for example Wilms' tumor or other tumors not originating from the kidney or non-malignant kidney pathologies. The areas of application of the invention include the field of medicine and the pharmaceutical industry but also basic biology. The aim of the invention is to provide novel forms of treatment which are urgently required regarding the present state-of-the-art in treatment of renal carcinoma and other cancers. It was discovered that the Nbk protein which is highly expressed in normal renal tissue, is either not expressed at all or only weakly expressed in the tumor tissue. The protein expression and the loss of Nbk was examined in renal cell carcinoma samples by the use of immunohistochemistry, PCR strategies, mutation and deletion analyses. The inventive method for diagnosis is characterized by that the Nbk protein concentration or the Nbk RNA quantity or Nbk mutation or deletion or Nbk gene modification, preferentially methylation, in tissue-derived material is determined. The inventive agent for the therapy of the renal carcinoma and other cells with low Nbk protein or RNA expression is that Nbk expression is increased and directly initiates the therapeutic effect in these cells. The implementation of a novel agent in non-tumorous kidney diseases is based on the inhibition of Nbk RNA or protein expression or Nbk activity.

The invention relates to a novel diagnostic and therapeutic approach in renal cell cancer and other renal tumors for example Wilms' tumor or other tumors not originating from the kidney and non-malignant tissues in various non-tumorous kidney pathologies. The areas of application of the invention include the field of medicine and the pharmaceutical industry but also basic biology.

So far, surgery is the only approach for the cure of non-metastatic renal cell cancer. As a standard procedure, radical tumor nephrectomy is performed including the regional lymph nodes. When such surgical therapy is applied, the 5-year overall survival of local tumors restriced to the kidney organ is 67 to 92%, depending on the local tumor and nodal disease stage. The disease prognosis of metastatic (stage N1 and higher or M1 according to the TNM classification) is far worse. In the case of such disseminated disease, the median overall survival is close to 6 months after diagnosis of metastatic disease and the 1-year overall survival is 28%. As compared to other malignant tumors, renal carcinoma is extremely resistant and does in general not respond to chemo-, radio- or hormonal therapy. Therefore, a cure of advanced renal cell cancer is so far impossible. Limited success was achieved more recently by the use of immunotherapy with interleukin-2 and/or interferons. Such immunotherapies may result in an overall response rate between 10 to 30% with a rather low rate of complete remissions as defined by clinical staging and imaging procedures. Since residual tumor cells and tissue survive immunotherapy, there is no convincing benefit from these therapies, i.e. prolongation of survival. Therefore, new methods for diagnosis and therapy of renal cell carcinoma are urgently needed to improve early diagnosis and better response to therapy.

The invention addresses the task to provide such novel diagnostic tools and to provide a novel approach for the treatment of renal cancer.

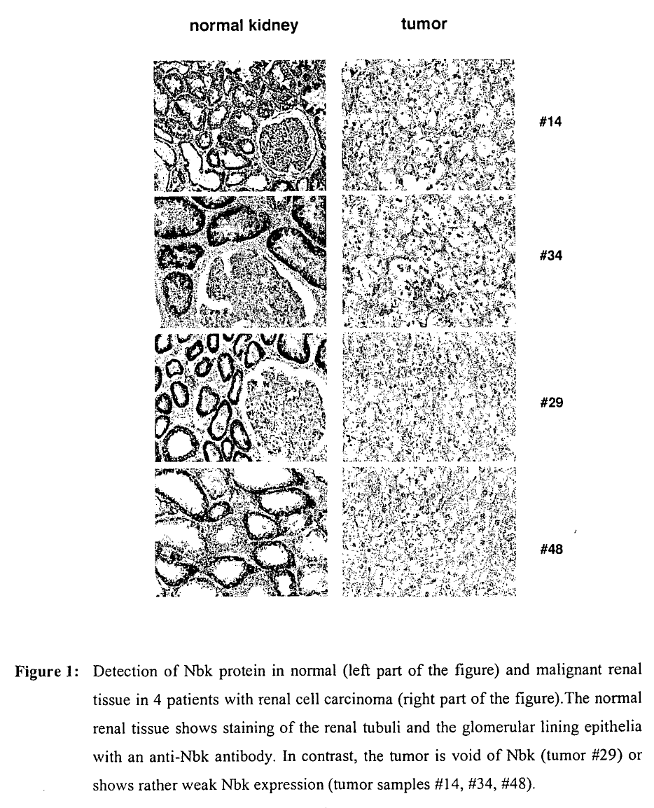

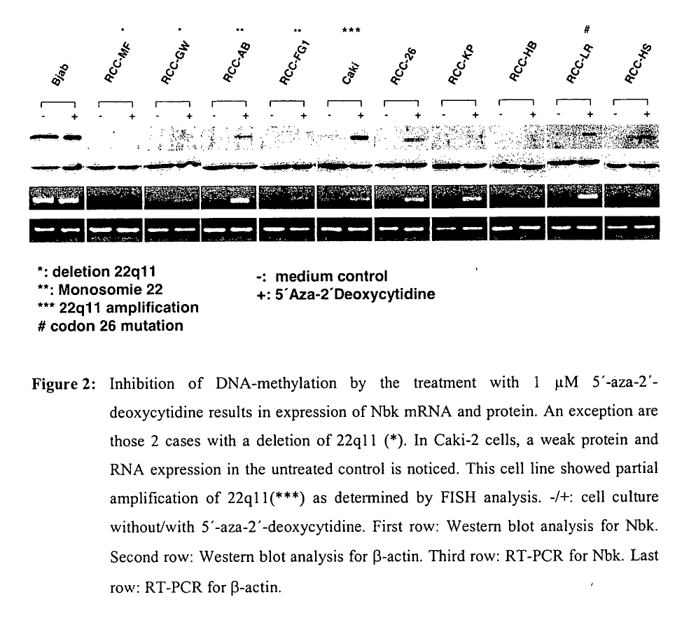

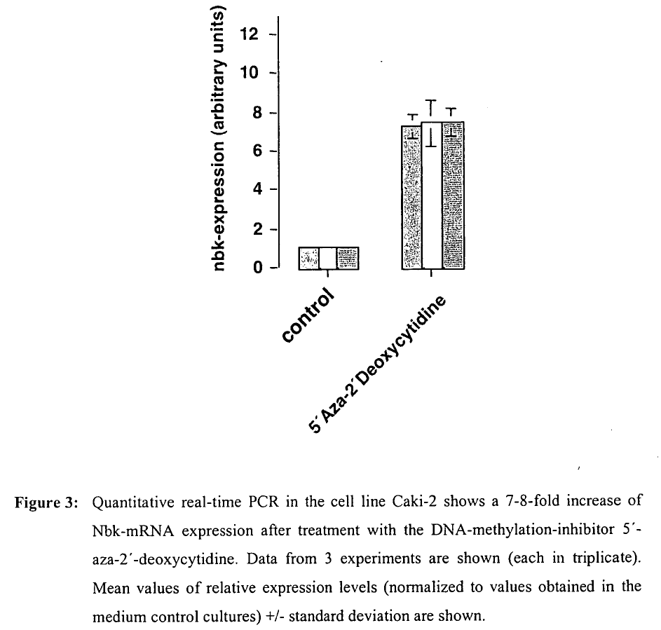

The knowledge regarding genetic abberrations in renal cell cancer does so far not allow a reasonable explanation for the extreme resistance to conventional and experimental cancer therapy. To obtain new insights into the underlying mechanisms, we examined a panel of genes known to have prognostic relevance in other cancers, including e.g. p53, Bax, p16. In addition, we included by chance the Nbk protein and its gene for which such a prognostic or pathogenetic relevance was not yet established. To our surprise, we found a high expression of Nbk protein in normal renal tissue both on the level of RNA and protein (FIG. 1). In contrast, renal cell carcinoma cells show either complete loss of Nbk protein and RNA expression or rather low level expression (FIGS. 2 and 3). The loss of Nbk was investigated in 80 samples of primary renal cell cancer tissue and a panel of renal cancer cell lines by the use of immunohistochemistry, mutation analysis based on the use of single strand conformational polymorphism polymerase chain reaction (SSCP-PCR), DNA sequencing, analysis of genetic deletion by fluorescence in situ hybridization (FISH), semiquantitative RT-PCR and quantitative real-time PCR. From these analyses, we conclude that inactivation of Nbk in renal cell cancer is a general pathomechanism that may explain the clinical and experimental resistance of renal cell cancer and other tumors that may have lost Nbk protein or RNA expression.

To explore the underlying reason for the loss of Nbk protein expression, a detained analysis of the Nbk gene was performed. Of the known 5 exons of the Nbk gene on chromosome 22q13 [1], exons 2 to 5 were analysed by the use of SSCP-PCR in DNA samples of the above mentioned renal cell cancer tumors and cell lines. In 10 patients, an aberrant band was detected in the SSCP-PCR. These samples were suspect for an Nbk mutation and were analysed, together with non-suspect samples by the use of DNA sequencing for the presence or absence of aberrations in the genomic DNA sequence. In addition, a panel of renal carcinoma cell lines was analyzed for Nbk mutation by sequencing of the complete Nbk cDNA. In contrast to an earlier observation in colorectal cancer [2], variations of the genetic code, i.e. single nucleotide polymorphisms, were found in the renal carcinoma samples and cell lines. Some of these were located in intronic sequences that may affect splicing of the mRNA, i.e. close to the intronic splice site, and consequently the expression of the Nbk protein.

Finally, Nbk was found to be lost in some tumor cells due to deletion of the 22q13 chromosomal region. This Nbk gene deletion correlated well with loss of Nbk mRNA and protein expression (FIG. 2).

Regarding downregulation of both Nbk mRNA and protein, the effect of DNA demethylation was studied. DNA methylation is a well known mechanism that is critical for the expression of the mRNA and the protein of a specific gene, i.e. transcriptional regulation and silencing. Culture of renal cell carcinoma cells was performed in the presence of a methylation inhibitor, 5′-aza-2′-deoxycytidine. Culture of tumor cells with impaired Nbk mRNA and protein expression in the presence of this methylation inhibitor led to an increase of Nbk RNA as determined by semi-quantitative PCR and protein as determined by Western blot analysis (FIG. 2). To quantify the increase of Nbk mRNA expression, quantitative real-time PCR was performed. Data obtained in Caki-2 cells are shown in FIG. 3. Real-time PCR quantitatively confirmed that DNA demethylation by the use of 5′-aza-2′-deoxycytidine strongly increases Nbk mRNA expression.

To further explore the significance of Nbk loss in tumor biological and to explore a therapeutic application based on complementation of Nbk expression by introduction of exogenous Nbk, an adenoviral vector was construced as described below and elsewhere [3]. The resulting Nbk adenovirus allows for the expression of the Nbk cDNA in renal cell carcinoma and this results in induction of apoptotic cell death.

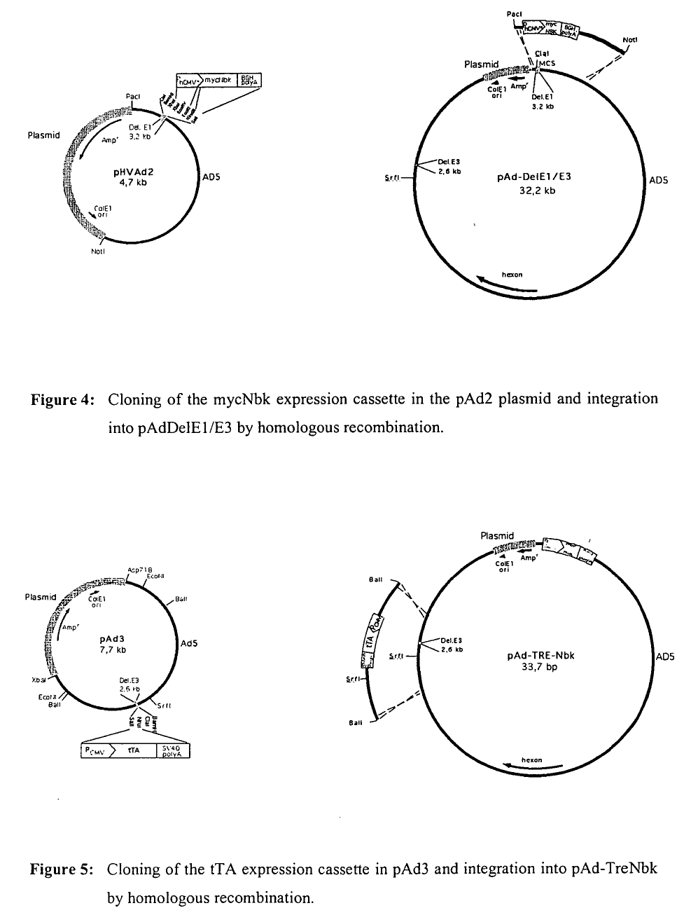

During construction of the Nbk adenoviral vector, technical problems had to be overcome. Construction of a conventional adenoviral vector based on the adenoviral serotype 5 backbone and a CMV promoter to drive Nbk expression failed as the the vector producing HEK293 cell line, a human embryonal kidney-derived cell line, consistently and repeatedly died upon transfection with the Nbk adenovirus DNA. Despite of variations of the procedure, no adenovirus for Nbk expression could be generated due to this inherent problem. Therefore, a technical development became necessary to allow for Nbk adenovirus generation and a conditional expression of the Nbk transgene upon use of the expression vector. To this end, a viral vector was to be constructed that allows for switching off Nbk expression during viral vector production as a fundamental basis for the generation of intact Nbk adenovirus vectors. Moreover, such a on-/off-switchable conditional expression system alows for a better control of the therapeutic transgene. To achieve this, the Tet-off system was introduced into the E1 and E3 regions of the adenoviral serotype 5 backbone (see below, FIG. 4 to 6).

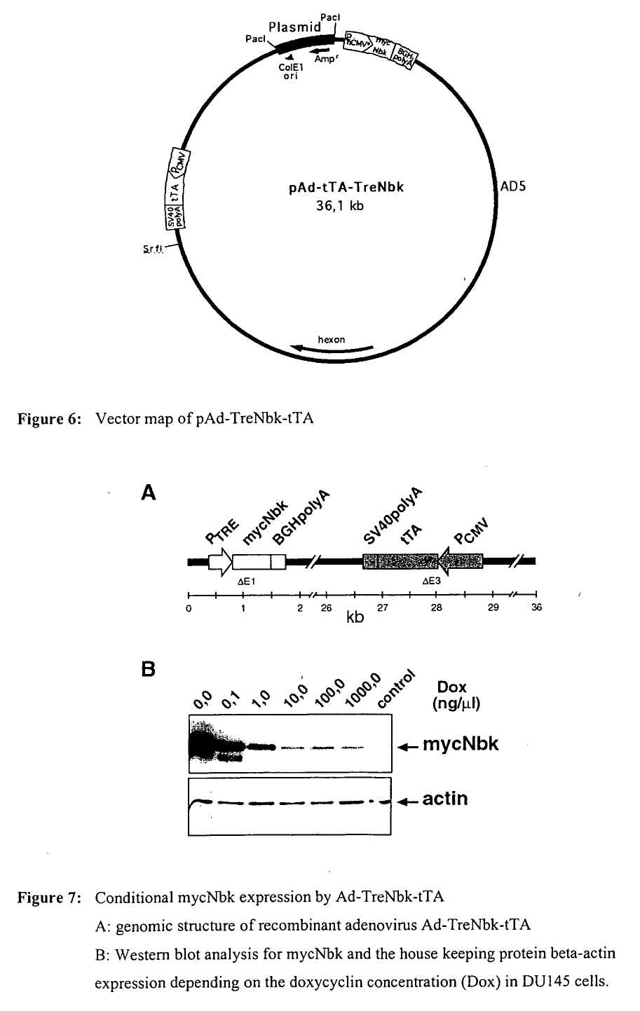

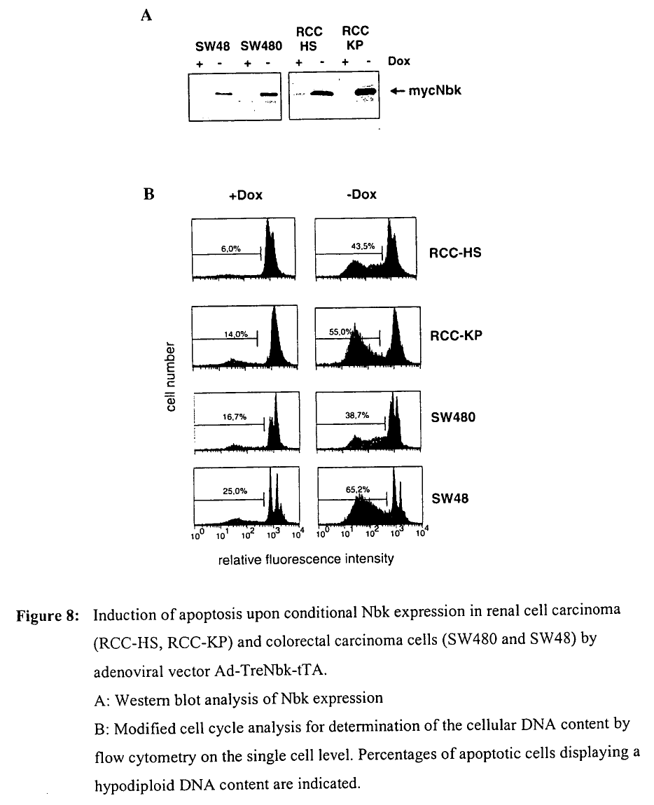

The adenoviral gene transfer of exogenous Nbk (FIG. 7) into a panel of renal cell carcinoma cells (Caki-2, RCC-FG1, RCC-HS, RCC-KP, RCC-LR, RCC-MF, RCC-26 and others) and non-renal cell carcinoma cells that are void of endogenous Nbk [3] proved as a surprisingly efficient tool to trigger cell death by apoptosis in the adenovirally transduced cells (FIG. 8). This is in clear contrast to earlier findings where ectopic and high overexpression of Nbk by the use of a pcDNA3 vector driven by a CMV promoter led to sensitization for apoptosis but did not result in induction of apoptosis per se [4]. In control experiments in tumor cells of other carcinomas where the non-malignant counterpart expresses Nbk whereas the malignant cell has lost Nbk expression [1], re-expression of Nbk by the use of the adenoviral vector for Nbk expression resulted also in induction of apoptotic cell death [3]. These cells were derived from colorectal cancer such as SW48 and SW480 cells (FIG. 8) and prostate cancer cells such as DU145 [3]. Therefore, Nbk gene transfer, e.g. by the use of an adenoviral vector, or re-expression of exogenous Nbk expression, e.g. by the use of Nbk gene methylation inhibitors or interference with proteasomal degradation [5] or other modifiers of gene silencing, represents a widely applicable therapeutic principle for the killing of tumor cells.

The invention is realized according to claims 1, 4, and 13. The subclaims represent preferential variants.

The invention is described in further detail by the following examples of implementation.

Abbreviations:

P_{CMV}: complete “immediate early” promoter of cytomegaly virus (CMV) that mediates efficient gene expression
P_{miniCMV}: minimal immediate early promoter of CMV that lacks the CMV enhancer and is therefore inactive in the absence of tTA binding to the TRE element
P_{hCMV*}: combined promoter that carries the TRE element directly in front of PminicMv.
TRE: tetracyclin responsive element, a regulatory sequence consisting of 7 copies of the tet operator (42 base pairs), a cis-regulatory DNA sequence of the bacterial tet operon that corresponds to the binding site of the tet repressor/transactivator tTA.
tTA: tetracycline controlled transactivator, a fusion protein consisting of TetR and the VP16 activation domain (VP16AD) of the herpes simplex VP16 protein.
BgHpolyA: bovine growth hormone polyadenylation signal
Amp^{R}: ampicillin resistance gene (beta lactamase)
ColiE1ori: high copy number origin of replication of E. coli
   Construction of a Conditional Adenoviral Vector System for the Adjustable Overexpression of Nbk

For regulable expression of the pro-apoptotic gene Nbk in human cells, an adenoviral vector based on the adenovirus serotype 5 backbone was constructed. In this construct, the E3 region was replaced by the expression cassette for the tTA and the E1 region was replaced by an expression cassette for Nbk. To allow for convenient detection of the exogenous Nbk, a myc tag was inserted at the N-terminus of the human Nbk cDNA. Constitutive expression of the tTA was achieved by putting the tTA under the control of a CMV promoter. In the absence of tetracyclin or doxycyclin the tTA fusion protein binds via the TetR portion to the TRE in the P_{hCMV*} promoter and activates via the VP16AD portion the transcription of the mycNbk. In the presence of tetra- or doxycyclin Nbk expression is repressed and switched off.

Vector construction was performed by the use of the following plasmids:

pAd1-Del-E1/E3

pAd1-Del-E1/E3 contains an adenovirus serotype 5 genome where both the E1 and the E3 region are deleted (FIG. 4). The E3 deletion corresponds to that described beforehand [6] from BHG10 and spans from 78.3 to 85.8 mu. Generation of the construct was performed by the use of homologous recombination in E. coli: The Pac1 restriction site of BHG10 was filled in and a 8.7 kb Hapl-NotI fragment was recombined with pTG3602 [7]. The E1 deletion spans from 0.9 to 9.8 mu and was introduced into the construct pAd1 by homologous recombination of a 2.7 kb PacI-NotI fragment of pAd2 (see below) that was linearised by the use of the ClaI restriction enzyme.

pAd2

The left-end shuttle plasmid pAd2 (FIG. 4) was employed for the insertion of genes into the E1-region. It contains 2.6 kb of the 5′ end of the adenovirus serotype 5 carying a 3.2 kb deletion in the E1 region. This largest possible E1 deletion E1 deletion and the polycloning site correspond to that employed beforehand [6] in pΔE1sp1A and the therein described deletions.

pAd3

The shuttle plasmid pAd3 (FIG. 5) was generated for the insertion of genes into the E3 region. It contains a 4.6 kb BgIII fragment of BHG10 [6]. This corresponds to 2.9 kb downstream and 1.7 kb upstream of the sequences of E3 deletions described earlier [6].

PAd-TreNbk-tTA

For construction of pAd-TreNbk-tTA (FIG. 6), the conditional tetracyclin responsive promoter element of pTre was obtained as XhoI/EcoRI fragment. This fragment was cloned into pAd2 by the use of the corresponding restriction sites in the shuttle plasmid. Then, Nbk cDNA was fused in frame with a myc tag at the 5′end. This mycNbk fragment including a BgH polyA signal was inserted into pAd2 by the use of the HindIII and SalI restriction sites (FIG. 4).

The thereby generated Nbk expression cassette (P_{hCMV*}; mycNbk; BgHpolyA) including the flanking adenoviral sequences was rescued as PacVNotI fragment and inserted into the pAd-DelE1/E3 after linearisation of the viral construct by use of the ClaI site (FIG. 4). Next, the expression cassette for the tTA was integrated into the thereby created plasmid pAd-TreNbk. To this end, the cassette containing a P_{CMV} promoter, the open reading frame for the tTA and a SV40 polyA signal was isolated from pTet-off as XhoI/PvuII fragment and cloned via use of the SalI and NruI restriction sites into the shuttle plasmid pAd3 (FIG. 5). From the resulting plasmid pAd3-tTA, the tTA cassette including the flanking adenoviral sequences was rescued as a Nhe/StuI fragment. This cassette was then introduced into the pAd 1-TreNbk by homologous recombination (FIG. 5). Thus, the resulting plasmid pAd-TreNbk-tTA contains a complete adenovirus serotype 5 genome (except the E1 and the E3 region) in which the E1 region was replaced by the Nbk expression cassette and the E3 region by tTA cassette (FIG. 6). The recombinant adenoviral genome of Ad-TreNbk-tTA was obtained by digestion and linearisation with the PacI restriction enzyme and was employed for transfection of HEK293 cells. In these cells that supply the adenoviral genes required for virus production in trans, the resulting Nbk adenovirus was propagated in the presence of doxycyclin to transcriptionally repress expression of the Nbk transgene during vector production.

Conditional Expression of Nbk in Human Carcinoma Cells

The recombinant adenovirus Ad-TreNbk-tTA (FIG. 7A) was employed for expression of the myc tagged Nbk (mycNbk) in a doxycyclin dependent manner. FIG. 7B shows protein expression of Nbk 24 h after infection with an Ad-TreNbk-tTA virus titer of 25 MOI (multiplicity of infection) in DU145 prostate carcinoma cells. In the presence of doxycyclin, Nbk expression by the vector was strongly suppressed and this suppression was alleviated upon decrease of the doxycyclin concentration. Nbk expression is almost completely suppressed at doxycyclin concentrations of 10 ng/ml or higher (FIG. 7B). Apart from DU145, Nbk could be strongly expressed by the use of this adenoviral vector 24 h after transduction in a panel of other carcinomas or non-epithelial tumor cells, i.e. sarcoma, glioma and other mesenchymal cell types.

Induction of Apoptosis by Ad-TreNbk-tTA-Mediated Expression of Nbk and Inhibition of Tumor Growth

The adenoviral expression vector Ad-TreNbk-tTA was employed to achieve strong expression of mycNbk and induction of apoptotic cell death in dependence from the concentration of doxycyclin. Western blot analysis demonstrates strong expression of mycNbk in cell lines cultured 24 h after transduction with Ad-TreNbk-tTA in the absence of doxycyclin (FIG. 8A). Apoptosis was determined by the use of a modified cell cycle analysis technique that relies on the measurement of cellular DNA content on the single cell level by flow cytometry. Such analyses show the occurrence of hypodploid (sub-G1) cells that have fragmented their genomic DNA, i.e. represent apoptotic cells, upon conditional expression of Nbk following gene transfer of Nbk by the use of Ad-TreNbk-tTA (FIG. 8B). Thus, conditional expression of Nbk induces apoptosis in the investigated renal carcinoma cells and a panel of other cell lines, e.g. colorectal or prostate cancer cells that have lost Nbk expression as compared to their non-malignant counterparts. Apart from the cells shown in FIG. 8B, apoptosis was induced by Ad-TreNbk-tTA in the absence of doxycyclin, i.e. the “on” setting of the expression vector, in the following cancer cells of renal origin RCC-LR, RCC-FGI, RCC-MF, RCC-26, Caki-2), in osteosarcoma cells (U2OS, Saos2), melanoma (MeI-2A, MeWo), colorectal and prostate carcinoma cells [3], a panel of malignant glioma cells [5] and in Matu breast carcinoma cells displaying resistance to anticancer drugs including adriamycin (Matu-Adr). Like in the renal cancer cells shown in FIG. 8B, Nbk induces massive apoptosis upon tranduction by the use of the Ad-TreNbk-tTA adenoviral vector in the absence of doxycyclin.

In the vein of these in vitro data, adenoviral gene transfer into the LN229 glioma cell line efficiently inhibited orthotopic tumor growth in a murine xenotransplant model in vivo [5]. Adenoviral gene transfer led to a significant improvement of the overall survival of tumor bearing mice. This anti-tumor effect is concordant with the propensity of Ad-TreNbk-tTA to induce apoptotic cell death in a panel of 12 glioma lines that lack endogenous Nbk expression in vitro as described [5].

Measurement of Nbk Protein Expression

Immunohistochemistry for Nbk/Bik-expression was performed in paraffin embedded tissue samples of human renal cell carcinoma. For protein detection by immunohistochemistry, 4 μm tissue slices were stained in a routine procedure after antigen demasking as described [8]. Primary antibody was an affinity-purified goat polyclonal antibody raised against a peptide corresponding to amino acids 95-114 mapping at the carboxy-terminus of Nbk of human origin. Analysis of slides was done blinded, without knowledge of clinicopathological data. Four microscope high power fields (400×enlarged) were evaluated for localization, percentage positive cells (0-100%) and staining intensity (0, i.e. negative to 3, i.e. strong expression). The product of percentage positive cells and intensitiy was calculated as the staining index (STI).

Non-tumorous renal tissue showed strong Nbk/Bik expression in the renal tubuli and the epithelial lining of the glomerula of normal kidney (FIG. 1). In contrast, only weak or rather complete loss of expression was seen in the renal cancer tissues. Comparison of normal renal tissue with cancer tissue in paired samples on the same slide was feasible in 28 cases, and the difference of expression was highly significant (staining index 221.4 (+/−5.4; standard error of the mean) in normal renal tissue and 31.1 (+/−60.3; standard error of the mean) in RCC tissue, p<0.0001 by Wilcoxon signed rank test).

For immunoblotting as employed below in the methylation experiments, standard procedures were applied as described [3]. Per lane, 20 μg protein was separated. Primary antibodies for immunoblotting were a polyclonal goat anti-Nbk antibody and a polyclonal goat anti-Nbk antibody raised against an epitope mapping at the C-terminus of human Nbk as employed for immunohistochemistry.

Assessment of the Genetic Status of the Nbk Gene

SSCP-PCR and DNA Sequencing

From 52 renal carcinoma samples, DNA was extracted from 30 μm slices of paraffin-embedded tissue, as described [8]. The coding exons 2 to 5 of the Nbk/Bik gene were amplified using intronic primers derived from consensus sequences AL022237.1, AF174421.1, AF174422.1, AF174431.1, and AF174441.1. Primer sequences and annealing temperatures were the following:

TABLE 1
Primer sequences and conditions for SSCP-PCR

| | | | Annealing | Product |
|---|---|---|---|---|
| Exon | | Primer Sequence | Temperature | size |
| 2 | Sense | TTA GGG GTC CAG TCA TAT GC | 55° C. | 253 bp |
| 2 | Antisense | CCT GAA GTC ACT ATC AGG CA | | |
| 3 | Sense | CGG CAC AGC CAC ACC CGA CT | 57° C. | 169 bp |
| 3 | Antisense | TGT AGA GGC ATA GGG CAT AG | | |
| 4 | Sense | CTC CTG CAG TAA TGG CTT TGT C | 60° C. | 202 bp |
| 4 | Antisense | TCA GGG TCA GGG ATC TCA AGG C | | |
| 5 | Sense | CTG CCC CGA GCC TGA CTC C | 67° C. | 174 bp |
| 5 | Antisense | TGG TCA TGG GGG TGG GGCC | | |

The genomic PCR was perfomed by the use of Taq DNA Polymerase applying standard conditions [8]. For SSCP analysis, fragments were diluted in denaturing loading buffer, boiled, cooled on ice, and analysed on a 10% non-denaturing polyacrylamide gel. Vizualisation was done by silver staining. Aberrant bands were identified as suspect for mutation and were confirmed by DNA sequencing applying the same primers as employed for the amplification of genomic Nbk DNA as described for p53 mutation beforehand [9]. SSCP-PCR and sequencing revealed two Nbk gene aberrations: a point mutation leading to an amino acid exchange at codon 26 (ACC->ATC, threonin>isoleucin) was found in 3 primary tumors and in the cell line RCC-LR. Furthermore, 48 tumors expressed an intronic CT deletion within the poly-pyrimidine tract upstream of the 3′splice site of intron 3. The CT-deletion was homozygous in 23 of the 52 tumors (SSCP PCR analysis was informative in 52 of 57 archival tumors), and heterozygous in 25 of the 52 tumors.

Fluorescence In-Situ Hybridization

Using bioinformatic resources clones RP3-323M22 (centromeric), CTB-1191B2 (spanning) and RP3-526114 (telomeric) were selected as FISH probes for the Nbk/Bik locus in 22q13.2. The cosmid clone 91c (AC000091; kindly provided by Bruce A. Roe; University of Oklahoma, Okla., USA) containing the TBX1 gene in the DiGeorge-critical region in 22q11.2 served as internal control. The differently labeled flanking probes were pooled to obtain a break-apart assay for the detection of breakpoints, the differently labeled clones CTB-1191 B2 and 91c were pooled to investigate genomic imbalances affecting the Nbk/Bik locus. The triple color probe set CEPX/CEPY/CEP18 was applied to study the general level of ploidy. Slides analysis revealed that Nbk gene deletion may occur but is a rather rare event.

Results from the FISH analyses and sequencing data from the renal carcinoma cell lines are depicted in relation to the re-expression of Nbk by the use of the methylation inhibitor 5′-aza-2-deoxycytidine Table 2:

TABLE 2
Nbk RNA and protein expression in relation to 5′-aza-2-deoxycytidine
(Aza) treatment and mutation/deletion status of the Nbk gene

| | | | Protein | | | |
|---|---|---|---|---|---|---|
| | RNA (RT-PCR) | | (Western Blot) | | Sequence | FISH |
| Treatment | −Aza | +Aza | −Aza | +Aza | Analysis | Analysis |
| RCC-MF | − | − | − | − | WT | del 22q11 |
| Caki-2 | ± | + | − | ++ | WT | amplifica- |
| | | | | | | tion of |
| | | | | | | 22q11 |
| RCC-26 | − | + | − | + | WT | |
| RCC-AB | − | + | − | + | WT | mono- |
| | | | | | | somy 22 |
| RCC-GW | − | ± | − | − | WT | del 22q11 |
| RCC-KP | − | + | − | + | WT | |
| RCC-FG1 | − | + | − | − | WT | mono- |
| RCC-HB | − | + | − | ± | WT | somy 22 |
| RCC-LR | − | + | − | + | codon 26 | |
| | | | | | A > T | |
| RCC-HS | − | + | − | + | WT | |

WT: wild type

Measurement of Nbk RNA Expression and the Effect of the Methylation Inhibitor 5-aza-2′-deoxycytidine or the Proteasome Inhibitor MG132

Repression of gene expression by hypemethylation of CpG islands in regulatory gene sequences has been described for several tumor suppresor genes, i.e. for the cyclin dependent kinase inhibitor p16^{INK4a} in various malignancies, for APAF-1 in melanomas, or for caspase-8 in neuroblastoma. We therefore treated 10 renal cell carcinoma cell lines with 5′-aza-2′-deoxycytidine (FIG. 2).

Semi-quantitative RT-PCR and Western blot analysis (FIG. 2) or quantitative real-time PCR (FIG. 3) were performed in cells that were treated with or without 5-aza-2′-deoxycytidine as inhibitor of DNA methylation. RNA extraction and RT-PCR from renal carcinoma cells was done according to standard procedures. For PCR, the following primer sequences were employed: Nbk sense GTC TGA AGT AAG ACC CCT CT, antisense ACT TGA GCA GCA GGT GCA GG; P-actin sense ACC CCC AAG GCC AAC CGC GAG AAG ATG ACC, antisense GGT GAT GAC CTG GCC GTC AGG CAG CTC GTA. PCR products were visualized by ethidium bromide staining. For quantitative real-time PCR, the following primers were employed: sense: TTA AGT GTG GTG AAA CCG TCC A, antisense: CAC AGC CTG GGT CTG GCT T, fluorescent probe: CAT CCC TGA TGT CCT CAG TCT GGT CGT labelled 5′ with 6FAM and 3′ with TAMRA.

Treatment with the methylation inhibitor 5′-aza-2′-deoxycytidine. resulted in significant expression of Nbk mRNA and protein. This was observed only in cells lacking Nbk gene deletion (FIG. 2). Induction of Nbk mRNA expression by 5′-aza-2′-deoxycytidine was confirmed by quantitative PCR in Caki-2 cells (FIG. 3). This indicates that Nbk expression may be lost in consequence of Nbk gene methylation.

Another technical implementation to achieve an increase in the levels of Nbk protein expression is the interference with proteasomal degradation of Nbk. Culture of glioma cells in the presence of the proteasome inhibitor MG132 enhanced Nbk expression levels upon adenoviral Nbk gene transfer by the use of Ad-TreNbk-tTA. This increase in Nbk expression correlated with an increased sensitivity for Nbk-induced apoptosis [5].

Likewise, increased expression of Nbk by the use of 5′-aza-2′-deoxycytidine resulted in an increased rate of apoptosis in treated cells. These data therefore establish that interference with Nbk expression, preferentially an increase of Nbk expresison in cancerous cells, is a feasible approach to trigger apoptotic death and demise of the targeted cell.

Role of Endogenous Nbk in Nbk Expressing Tissues

Nbk is an apoptosis inducing gene that is expressed in a tissue specific manner. According to the described data a regulatory, cell death promoting role in non-malignant cells displaying Nbk expression is deduced. Thus, the loss of Nbk expression as shown above in renal carcinoma appears to be a decisive step in the acquisition of resistance to apoptosis in renal carcinoma cells and therefore appears to represent a key step malignant transformation during tumorigenesis in the kidney and possibly also in other tissues displaying constitutive or inducible Nbk expression. Consequently, deregulated activity of such a central cell death activator is deleterious in non-malignant pathologies. These pathologies include hereditary syndromes like cystic kidney disease, kidney damage and failure in consequence of endogenous or exogenous toxins including bacterial toxins and heavy metals. The same applies for metabolic diseases including diabetes mellitus and hypoxic kidney damage. Moreover, evidence is presented by the above described data that viral infection leading to expression and activation of Nbk may result in apoptotic death of kidney cells. Finally, immune-mediated mechanisms may lead to renal damage via Nbk, e.g. in the case of infectious or non infectious nephritis, i.e. during viral, bacterial infection or kidney transplant-rejection.

Based on this model of Nbk as tissue specific mediator of renal damage another implementation of the invention was developed: to inhibit Nbk gene expression and activation at the transcriptional, the translational or the posttranslational level. This is achieved by the inhibition of the Nbk protein itself, e.g. by the use of peptides that block the interaction with Bax that is a key effector of Nbk-induced apoptosis [3] or by the use of pharmacological inhibitors such as small molecules that prevent Nbk interaction with Bax or other effectors of Nbk-induced apoptosis. Another application is the interference with Nbk-induced signalling events at the level of the mitochondria or the endoplasmic reticulum or other organelles. To this end, we expressed the anti-apoptotic Bcl-2 protein in DU145 cells. Expression of Bcl-2 was targeted to the mitochondria by the use of an actA signal peptide and to the endoplasmic reticulum (ER) by the use of a cytochrome b5-derived signal peptide as described in other systems beforehand [10]. Inhibition of Nbk could be achieved by either localization of Bcl-2, at the ER or at the mitochondria. This indicates that both organelles are critically involved in the execution of apoptosis by Nbk.

Likewise, interference with Nbk RNA expression by the use of siRNA or antisense oligonucleotides may interfere with the above mentioned pathologies that are linked to excess expression or activity of Nbk. This approach represents a novel therapeutic principle in the treatment of non-malignant kidney disease.

### Cited Literature

1. Venrna, S., Budarf, M. L., Emanuel, B. S., Chinnadurai, G. (2000) Structural analysis of the human pro-apoptotic gene Bik: chromosomal localization, genomic organization and localization of promoter sequences. Gene. 254, 157-62
2. Abdel-Rahman, W., Arends, M., Morris, R., Ramadan, M., Wyllie, A. (1999) Death pathway genes Fas (Apo-1/CD95) and Bik (Nbk) show no mutations in colorectal carcinomas. Cell Death Differ. 6, 387-8
3. Gillissen, B., Essmann, F., Graupner, V., Starck, L., Radetzki, S., Dbrken, B., Schulze-Osthoff, K., Daniel, P. T. (2003) Induction of cell death by the BH3-only Bcl-2 homolog Nbk/Bik is mediated by an entirely Bax-dependent mitochondrial pathway. EMBO J. 22, 3580-90
4. Radetzki, S., Köhne, C. H., von Haefen, C., Gillissen, B., Sturm, I., Dörken, B., Daniel, P. T. (2002) The apoptosis promoting Bcl-2 homologues Bak and Nbk/Bik overcome drug resistance in Mdr-1-negative and Mdr-1 overexpressing breast cancer cell lines. Oncogene. 21, 227-38
5. Naumann, U., Frank, B., Weit, S., Gillissen, B., Daniel, P. T., Weller, M. (2003) Adenoviral natural born killer gene therapy for malignant glioma. Human Gene Ther. 14, 1235-46
6. Bett, A. J., Haddara, W., Prevec, L., Graham, F. L. (1994) An efficient and flexible system for construction of adenovirus vectors with insertions or deletions in early regions 1 and 3. Proc Natl Acad Sci USA. 91, 8802-6
7. Chartier, C., Degryse, E., Gantzer, M., Dieterle, A., Pavirani, A., Mehtali, M. (1996) Efficient generation of recombinant adenovirus vectors by homologous recombination in Escherichia coli. J. Virol. 70, 4805-10
8. Sturm, I., Kohne, C. H., Wolff, G., Petrowsky, H., Hillebrand, T., Hauptmann, S., Lorenz, M., Dörken, B., Daniel, P. T. (1999) Analysis of the p53/BAX pathway in colorectal cancer: low BAX is a negative prognostic factor in patients with resected liver metastases. J Clin Oncol. 17, 1364-74
9. Sturm, I., Bosanquet, A. G., Hermann, S., Güner, D., Dörken, B., Daniel, P. T. (2003) Mutation of p53 and consecutive selective drug resistance in B-CLL occurs as a consequence of prior DNA damaging chemotherapy. Cell Death Differ. 10, 477-84
10. Rudner, J., Lepple-Wienhues, A., Budach, W., Berschauer, J., Friedrich, B., Wesselborg, S., Schulze-Osthoff, K., Belka, C. (2001) Wild-type, mitochondrial and ER-restricted Bcl-2 inhibit DNA damage-induced apoptosis but do not affect death receptor-induced apoptosis. J Cell Sci. 114, 4161-72

1. Method for the diagnosis of renal carcinoma and other kidney tumors including the wilms' tumor and other tumors, characterized by a measurement of the Nbk concentration in the tissue at the protein or RNA level.

2. Method according to claim 1, characterized by an analysis for Nbk gene alterations such as mutations and polymorphisms, preferentially by DNA sequence analysis and measurement of the Nbk concentration of the protein or RNA level.

3. Agent for the treatment of renal carcinoma and other renal tumors, for example the wilms' tumor, and other tumors not originating from the kidney characterized by causing an increase in the Nbk protein concentration in renal or other tissues and thereby mediating a therapeutic effect.

4. Agent according to claim 3, characterized by that it increases Nbk protein expression in consequence of a transfer of Nbk cDNA or the entire Nbk gene or parts of it.

5. Agent according to claims 3 and 4, characterized by that it increases the Nbk protein expression by use of viral or non-viral expression vectors.

6. Agent according to claims 3 to 5, characterized by that the Nbk protein expression is increased by use of a conditional adenoviral expression vector, preferentially by use of a vector as depicted FIG. 6, constructed by cloning the expression cassette in the plasmid pAD3 as shown in FIG. 3.

7. Agent according to claim 3, characterized by tissue specific expression of Nbk by preferential use of tissue specific promoters or other, e.g. hormonal or other pharmacological regulators.

8. Agent according to claim 3, characterized by tissue specific expression of Nbk that is achieved by the use of chimeric molecules consisting of Nbk and components of transcription factors or signal peptides at the level of the Nbk gene, the RNA or the protein.

9. Agent according to claim 3, characterized by the property to induce re-expression of downregulated endogenous Nbk, e.g. by use of pharmacological stimulation of Nbk protein expression by activating the gene expression and by activating regulation of the Nbk promotor.

10. Agent according to claim 3, characterized by increasing the Nbk protein expression by stabilisation of the Nbk protein expression, e.g. by interference with Nbk degradation.

11. Agent according to claim 3, characterized by that the Nbk activity is increased or inhibited by activation or inhibition of regulatory parts of the Nbk protein.

12. Agent for the therapy of hereditary or somatic renal diseases such as degenerative kidney disease, infectious and non-infectious inflammable or toxic renal damage in which cells die by apoptotic cell death, characterizied by that inhibitors of the expression or activity of Nbk protein or Nbk RNA are brought into renal cells.

13. Agent according to claim 12, characterized by that the activity of the Nbk protein or the Nbk RNA is decreased by activation or inhibition of regulatory parts of the Nbk gene.

14. Agent according to claim 12, characterized by that the activity of the Nbk protein or Nbk RNA is decreased by activation or inhibition of downstream signaling pathways.
